(19) United States
(12) Patent Application Publication
(10) Pub. No.: US 2016/0051565 A1
(43) Pub. Date: Feb. 25, 2016

(54) Transdermal Pharmaceutical Compositions Including Testosterone and an Aromatase Inhibitor

(71) Applicant: Professional Compounding Centers of America (PCCA), Houston, TX (US)

(72) Inventors: Tsu-I Catherine Wang, Sugar Land, TX (US); Bruce V. Biundo, Houston, TX (US)

(21) Appl. No.: 14/830,524

(22) Filed: Aug. 19, 2015

Related U.S. Application Data
(60) Provisional application No. 62/039,825, filed on Aug. 20, 2014.

(51) Int. Cl. A61K 31/568; A61K 47/10; A61K 9/00; A61K 31/138; A61K 45/06; A61K 31/4196

(52) U.S. Cl. CPC A61K 31/568; A61K 45/06; A61K 31/4196; A61K 9/0014; A61K 31/138; A61K 47/10

(57) ABSTRACT

Formulations for transdermal pharmaceutical compositions including a synergistic combination of low doses of testosterone with an aromatase inhibitor (AI) that are combined with transdermal permeation enhancers are disclosed. Transdermal pharmaceutical compositions can be designed with various release rates, and can be administered to increase bloodstream testosterone levels and thereby reduce symptoms of testosterone deficiency. Transdermal pharmaceutical compositions include liquid dosage forms, such as, for example solutions, liquid sprays, lotions, and the like. Additionally, transdermal pharmaceutical compositions include semi-solid dosage forms, such as, for example emulsions, creams, gels, pastes, ointments, and the like. Transdermal pharmaceutical compositions will deliver testosterone and AI through the skin and directly into the patient's bloodstream, thereby providing high bioavailability of testosterone and AI. The dosage regimen of the transdermal pharmaceutical compositions can be easily tailored for individual patients according to the baseline blood levels of testosterone and estradiol.

### CROSS-REFERENCE TO RELATED APPLICATIONS

This application claims priority to U.S. Provisional Application Ser. No. 62/039,825, filed Aug. 20, 2014, which is hereby incorporated by reference.

### BACKGROUND

1. Field of the Disclosure

The present disclosure relates generally to pharmaceutical compositions, and more particularly, to transdermal pharmaceutical compositions including testosterone synergistically combined with an aromatase inhibitor (AI) for testosterone deficiency and to maintain estradiol within normal physiologic levels.

2. Background Information

Testosterone is the androgenic hormone primarily responsible for normal growth and development of male sex and reproductive organs, including the penis, testicles, scrotum, prostate, and seminal vesicles. Testosterone facilitates the development of secondary male sex characteristics, such as musculature, bone mass, fat distribution, hair patterns, laryngeal enlargement, and vocal cord thickening, among others. Additionally, normal testosterone levels maintain energy level, healthy mood, fertility, and sexual desire.

The production of testosterone by the testes is regulated by a complex chain of signals that begins in the brain, mediated by the hypothalamic-pituitary-gonadal axis. The hypothalamus secretes gonadotropin-releasing hormone (GnRH) to the pituitary gland in pulses (bursts) which triggers the secretion of luteinizing hormone (LH) from the pituitary gland. Luteinizing hormone stimulates the Leydig cells of the testes to produce testosterone. Normally, the testes produce approximately 4 mg to 7 mg of testosterone per day.

Generally speaking, testosterone production declines naturally with age. In addition, low testosterone or testosterone deficiency (TD) may result from disease or damage to the hypothalamus, pituitary gland or testicles that inhibit hormone secretion and testosterone production. Testosterone deficiency is commonly referred to as hypogonadism. Depending on age, insufficient testosterone production can lead to abnormalities in muscle and bone development, underdeveloped genitalia and diminished virility.

Currently, the most common treatment for symptomatic male testosterone deficiency is testosterone supplementation with various transdermal, oral, buccal, and injectable delivery methods. These methods typically involve very high doses of testosterone. The main purpose of the testosterone replacement therapy is to achieve normal range of testosterone serum levels.

Current oral therapy of testosterone lacks effectiveness because testosterone is metabolized extensively during the first passage through the liver before reaching the systemic blood circulation (e.g., the first-pass effect). Intramuscular injections of testosterone are widely used, but severe drawbacks for this form of treatment include local pain, soreness, minor swelling, and the unphysiologically high levels of testosterone in the body during the first days/weeks after injection. Local pain is attributed to the large volumes of testosterone injected at a specific injection site. Other drawbacks of intramuscular injections include the need for required assistance of health care professionals thereby making injections inconvenient and expensive.

Additionally, testosterone replacement therapy can be associated with side effects, such as gynecomastia, nipple tenderness, and the like. Further, long-term testosterone replacement therapy will cause testicular atrophy and decline in sperm counts due to suppression of the hypothalamic-pituitary-gonadal axis via a negative feedback mechanism. Physiologic inhibition of pituitary gonadotropin secretion in men by testosterone is mainly mediated by aromatization to estrogen, which inhibits hypothalamic secretion of GnRH. Low levels of gonadotropin releasing hormone (GnRH) further decrease production of LH and follicle stimulating hormone (FSH) by the pituitary gland. The low LH levels translate to low testosterone production by the Leydig cells in the testes. The reduction in FSH often results in suppression of spermatogenesis. Therefore, there is a need for a testosterone replacement therapy that does not include the aforementioned side-effects.

### SUMMARY

The present disclosure refers to transdermal pharmaceutical compositions that include a synergistic combination of low doses of testosterone with an aromatase inhibitor (AI). Further, these transdermal pharmaceutical compositions are proposed to increase testosterone levels in a patient's bloodstream and reduce symptoms of testosterone deficiency. The synergistic combination of AI and low doses of testosterone may lead to increased levels of testosterone in the patient without the side effect of high estrogen levels. As such, transdermal pharmaceutical compositions can be used in treating a wide variety of conditions resulting from testosterone deficiency in men.

In some embodiments, APIs include low doses of testosterone synergistically combined with an AI, such as, for example anastrozole (Arimidex®), letrozole (Femara®), exemestane (Aromasin®), vorozole (Rivizor®), formestane (Lentaron®), fadrozole (Afema®), testolactone (Teslac®), or any other chemical compound that exhibits aromatase inhibition. In an example, the AI employed in transdermal pharmaceutical compositions is anastrozole.

In another example, the amount of anastrozole included within transdermal pharmaceutical compositions range from about 0.01% to about 0.1%. These percentages may refer to % weight by weight, % weight by volume, or % volume by volume.

In other embodiments, testosterone can be administered in the form of a testosterone ester. Examples of testosterone esters include testosterone cypionate, testosterone propionate, testosterone enanthate, testosterone heptylate, testosterone caproate, testosterone phenylpropionate, testosterone isocaproate, testosterone decanoate, testosterone acetate, testosterone laurate, or a pharmaceutically acceptable ester thereof, or any combination thereof.

In an example, the amount of testosterone included within transdermal pharmaceutical compositions range from about 2% to about 20%; preferably from about 5% to about 10%. These percentages may refer to % weight by weight, % weight by volume, or % volume by volume.

In some embodiments, various additives are included to facilitate the preparation of suitable dosage forms. For example, additives include diluents, thickening agents, transdermal penetration enhancers, pH adjusters, preservatives, colors, stabilizing agents, antioxidants, and surfactants, among others.

In some embodiments, transdermal penetration enhancers provide more efficient penetration of API through skin. In these embodiments, the required concentration of penetration enhancers depends on penetration enhancers' chemical properties and the API included within transdermal pharmaceutical compositions. Further to these embodiments, the transdermal penetration enhancers may allow lower API dosage requirements.

In an example, the amount of penetration enhancers included within transdermal pharmaceutical compositions range from about 0.5% to about 50%; preferably from about 1% to about 20%. These percentages may refer to % weight by weight, % weight by volume, or % volume by volume.

In some embodiments, transdermal pharmaceutical compositions allow the delivery of testosterone and AIs directly into the patient's bloodstream bypassing the gastrointestinal tract and the hepatic metabolism. In these embodiments, transdermal pharmaceutical compositions will provide higher percentages of bioavailability of testosterone and AIs to the patient, and this also allows lower dosage requirements for testosterone.

In some embodiments, transdermal pharmaceutical compositions include liquid dosage forms, such as, for example solutions, liquid sprays, lotions, and the like. In other embodiments, transdermal pharmaceutical compositions include semi-solid dosage forms, such as, for example emulsions, creams, gels, pastes, ointments, and the like.

In some embodiments, transdermal pharmaceutical compositions are applied to any area of skin, such as, for example planter foot arch, lateral ankle, palm, upper arm, ventral forearm, dorsal forearm, back, chest, thigh, abdomen, groin, scalp, axilla, forehead, lower back, buttocks or scrotum, among others. In these embodiments, most suitable sites to apply transdermal pharmaceutical compositions are ventral forearm, upper arm, and chest. In other embodiments, transdermal pharmaceutical compositions are applied to those areas of skin that provide maximal systemic absorption due to increased cutaneous blood flow and heat.

In an example, transdermal pharmaceutical compositions are administered within a dosage range of about 25 mg/day to about 500 mg/day of testosterone, preferably from about 40 mg/day to about 120 mg/day; and from about 0.01 mg/day to about 1.0 mg/day of anastrozole, preferably from about 0.1 mg/day to about 0.5 mg/day.

In some embodiments, transdermal dosage forms can be designed for fast release and transdermal absorption of testosterone and AIs. In other embodiments, transdermal dosage forms can be designed for slow release and transdermal absorption of testosterone and AIs over a prolonged period of time.

In some embodiments, low dose APIs in any of the above identified dosage forms can result in acceptable testosterone levels in the patient. This contrasts with conventional testosterone replacement therapy that involves administering high dosages of testosterone.

Numerous other aspects, features, and benefits of the present disclosure may be made apparent from the following detailed description.

### DETAILED DESCRIPTION

The present disclosure is here described in detail. Other embodiments may be used and/or other changes may be made without departing from the spirit or scope of the present disclosure. The illustrative embodiments described in the detailed description are not meant to be limiting of the subject matter presented here.

Definitions

As used here, the following terms have the following definitions:

“Absorption Enhancer” or, equivalently, “Penetration Enhancer” refers to a substance used to increase the rate of permeation through the skin or other body tissue of one or more substances (e.g., APIs) in a formulation.

“Active Pharmaceutical Ingredients (APIs)” refer to chemical compounds that induce one or more desired effects that are therapeutically or prophylactically effective.

“Aromatase Inhibitors (AIs)” refers to chemical compounds that block or inhibit the activity of aromatase which is an enzyme that converts androgens to estrogens. As such, an aromatase inhibitor acts to reduce estrogen levels in the body.

“Permeation enhancement” refers to an increase in the permeability of a selected active pharmaceutical ingredient (API) through the skin.

“Transdermal drug delivery” refers to administration of a drug to the skin surface of an individual so that the drug passes through the skin tissue and into the individual's bloodstream, thereby providing a systemic effect.

“Treating” and “Treatment” refer to reduction in severity and/or frequency of symptoms, elimination of symptoms and/or underlying cause, prevention of the occurrence of symptoms and/or their underlying cause, and improvement or remediation of damage.

“Vehicle” refers to a substance of no therapeutic value that is used to convey at least one API for administration.

Description of the Disclosure

Embodiments of the present disclosure are directed towards transdermal delivery of active pharmaceutical ingredient (APIs). Transdermal pharmaceutical compositions that include synergistic combinations of AI with low doses of testosterone as APIs are disclosed. Further, these transdermal pharmaceutical compositions are proposed to increase testosterone levels, maintain estradiol levels within physiologic range, and reduce symptoms of testosterone deficiency in men without the side effect of high estrogen levels.

Transdermal drug delivery is receiving increased attention because it can provide a controlled release rate of active pharmaceutical ingredients (APIs) into the systemic circulation of the patient. The delivery of APIs through the skin provides many benefits. Primarily, such means of delivery is a comfortable, convenient and non-invasive way of administering APIs. The first-pass metabolism associated with oral administration is avoided, and other inherent inconveniences, such as gastrointestinal irritations, are eliminated as well.

Transdermal delivery is a particularly advantageous delivery route. It is a non-invasive drug delivery method with the benefits of better patient compliance, less risk of infection, and lower cost than invasive procedures, such as injection and implantation. Transdermal delivery may also provide a much shorter onset time (e.g., the time from administration to therapeutic effect) than oral delivery does. Transdermal applications of APIs are simple and can be administered by a caregiver or the patient with minimal discomfort.

Recently, research studies have demonstrated that aromatase inhibitors (AIs) as APIs may be used to treat low testosterone levels in men. AIs work by binding to the aromatase and inhibiting this enzyme that converts testosterone into estrogen. Estradiol serves as a major mediator of sex steroid-gonadotropin feedback; hence, high estradiol levels could contribute to low testosterone production through inhibition of LH. Meanwhile, high estradiol levels can also exist independently of testosterone levels. AIs effectively inhibit or block conversion of testosterone into estrogen which leads to increased LH and follicle-stimulating hormone (FSH) release from the pituitary gland. Increased LH and FSH results in a subsequent increase in testicular stimulation and serum testosterone levels without the increase in estrogen levels, and thus could limit the likelihood of undesirable effects, such as gynecomastia.

Formulation

In some embodiments, transdermal pharmaceutical compositions include a synergistic combination of AI and low doses of testosterone as APIs, transdermal penetration enhancers, vehicles, and additives, among other suitable ingredients. In these embodiments, APIs include low doses of testosterone synergistically combined with an AI, such as, for example anastrozole (Arimidex®), letrozole (Femara®), exemestane (Aromasin®), vorozole (Rivizor®), formestane (Lentaron®), fadrozole (Afema®), testolactone (Teslac®), or any other chemical compound that exhibits aromatase inhibition. In an example, the AI employed in transdermal pharmaceutical compositions is anastrozole.

In another example, the amount of anastrozole included within transdermal pharmaceutical compositions range from about 0.01% to about 0.1%. These percentages may refer to % weight by weight, % weight by volume, or % volume by volume.

In some embodiments, testosterone can be administered in the form of a testosterone ester. Examples of testosterone esters include testosterone cypionate, testosterone propionate, testosterone enanthate, testosterone heptylate, testosterone caproate, testosterone phenylpropionate, testosterone isocaproate, testosterone decanoate, testosterone acetate, testosterone laurate, or a pharmaceutically acceptable ester thereof, or any combination thereof.

In an example, the amount of testosterone included within transdermal pharmaceutical compositions range from about 2% to about 20%; preferably from about 5% to about 10%. These percentages may refer to % weight by weight, % weight by volume, or % volume by volume.

In some embodiments, various additives are included to facilitate the preparation of suitable dosage forms. For example, additives include diluents, thickening agents, transdermal penetration enhancers, pH adjusters, preservatives, colors, stabilizing agents, antioxidants, and surfactants, among others.

In some embodiments, a pH adjusting agent includes sodium bicarbonate, sodium hydroxide, magnesium hydroxide, calcium carbonate, calcium hydroxide, dibasic calcium phosphate, tribasic calcium phosphate, potassium hydroxide, citric acid, lactic acid, hydrochloric acid, sulfuric acid, phosphoric acid, sodium phosphate monobasic, sodium phosphate dibasic, diethanolamine, and triethanolamine, among others.

In some embodiments, surfactants include: polysorbates, such as, for example polysorbate 20, 40, 60, and 80, among others; sorbitan esters, such as, for example sorbitan monolaurate, and sorbitan monopalmitate, sorbitan monooleate, among others; and sodium lauryl sulfate, among other surfactants known to those skilled in the art.

In some embodiments, a stabilizing agent is used to stabilize the API for a specific dosage form. In these embodiments, the stabilizing agent used will depend on the API used as well as the other additive ingredients. Any suitable chemical substance may be used as a stabilizing agent. Stabilizing agents are known to those skilled in the art and therefore will not be discussed further herein.

In some embodiments, solvents for liquid dosage forms of transdermal pharmaceutical compositions include water, liquid polyethylene glycols of various molecular weights, ethyl oleate, medium chain triglycerides, isopropyl myristate, isopropyl palmitate, isopropyl stearate, other pharmaceutically acceptable esters of C8-C22 fatty acids and C2-C6 alcohols, mineral oil, and vegetable oils, among others.

In some embodiments, transdermal penetration enhancers provide more efficient penetration of API through skin. In these embodiments, the required concentration of penetration enhancers depends on penetration enhancers' chemical properties and the API included within transdermal pharmaceutical compositions. Further to these embodiments, the transdermal penetration enhancers may allow lower API dosage requirements.

In some embodiments, transdermal penetration enhancers include: physical enhancers, such as, for example iontophoresis, sonophoresis, phonophoresis, magnetophoresis, electroporation, thermophoresis, radio frequency, needleless injection, hydration of stratum corneum, and stripping of stratum corneum, among others; alcohols including alkanols and alkenols, such as, for example ethanol, 1-octanol, 1-hexanol, 1-decanol, lauryl alcohol, linolenyl alcohol, and pentylene glycol, among others; alkyl-N,N-disubstituted amino acetates, such as, for example dodecyl-N,N dimethylaminoacetate, and dodecyl 2-(dimethyl amino) propanoate derivatives, among others; azone analogs with different polar heads and hydrophobic chain length, such as, for example azone, 1-alkyl or 1-alkenylaza cycloalkanones, among others; ceramide analogs with different polar heads and hydrophobic chain length; cyclodextrins (form complex with APIs and increase the absorption in the presence of other transdermal penetration enhancers); essential oils, such as, for example ajuput oil, Alpinia oxyphylla oil, anise oil, basil oil, cardamom oil, chamomile oil, chenopodium oil, citronella oil, black cumin oil, clove oil, Eryngium bungei essential oil, eucalyptus oil, fennel oil, ginger oil, lilacin oil, lavender oil, menthe oils, melissa oil, myrtle oils, neem oil, niaouli oil, nutmeg oil, orange oil, peppermint oil, petit grain oil, rosemary oil, sage oil, turpentine oils, tulsi oil, thyme oil, tea tree oil, and ylang-ylang oil, among others; fatty acid esters, such as, for example cetyl lactate, butyl acetate, and isopropyl myristate, among others; fatty acids, such as, for example capric acid, caprylic acid, cis 11, 14-eicosadienoic acid, oleic acid, lauric acid, linoleic acid, linolenic acid, margaric acid, myristic acid, palmitic acid, and stearic acid, among others; propylene glycol conjugates of unsaturated fatty acids; glycols, such as, for example propylene glycol, polyethylene glycol 400, and glycerols, among others; oxazolidinones, such as, for example 4-decyloxazolidine-2-one and 3-acetyl-4-decyloxazolidin-2-one, among others; pyrrolidones, such as, for example 2-pyrrolidone, N-methyl-2-pyrrolidone, and 1-lauryl-2-Pyrrolidone, among others; sulfoxides and similar compounds, such as, for example dimethylsulfoxide, dimethylacetamide, and dimethyl formamide, among others; surfactants, such as, for example sodium lauryl sulphate, sorbitan monopalmitate, sorbitan trioleate, cetyl trimethyl ammonium bromide, benzalkonium chloride, and dodecyl betaine, among others; saponins and other herbal extracts, such as, for example Glycyrrhiza glabra, glycyrrhizin, Asparagus racemosus, Aloe vera, Quillaja saponaria, Acanthophyllum squarrusom, Coptis japonica and its alkaloidal isolates (berberine, coptisine, and palmatine), and Senkyu (Ligustici Chuanxiong Rhizome) ether extract, among others; terpenes and terpenoids, such as, for example alpha-terpinol, alpha terpineol, alpha pinene, ascaridol, alpha bisabolol, cavacrol, carvone, 1,8 cineole, p-cymene, eucalyptol, farnesol, fenchone, geraniol, limonene, limonene oxide, linalool, menthol derivatives, thiomenthol derivatives, o-ethylmenthol derivatives, menthone, neomenthol, nerolidol, pulegone, terpinen-4-ol,tetrahydrogeraniol, thymol, trans-anethole, and verbenone, among others; transcarbam 12 derivatives, such as, for example 5-(dodecyloxycarbonyl)pentylammonium-5-(dodecyloxycarbonyl)pentylcarbamate, and iminosulfurane, such as, for example N-hexyl,N-benzoyl-S,S-dimethylimino-sulfuranes, among others; capsaicin derivatives, such as, for example nonivamide; cinnamene compounds, such as, for example cinnamic acid, cinnamaldehyde and cinnamic alcohol, among others; tranexamic acid derivatives; or urea and derivatives, such as, for example urea, 1-dodecylurea, 1-dodecyl-3-methyl urea, 1-dodecyl-3-methylthiourea, and cyclic urea derivatives, among others.

In other embodiments, transdermal penetration enhancers include: lipid synthesis inhibitors, such as, for example 5-tetradecyloxy-2-furancarboxylic acid, fluvastatin, and cholesterol sulfate, among others; phospholipids, such as, for example phosphatidyl choline from egg yolk and soybean, dimyristyl phsphatidyl glycerol, dipalmityl phophatidyl glycerol, distearyl phosphatidyl glycerol, dioleyl phosphatidyl glycerol derivatives, phosphatidyl choline derivatives from soybean and egg yolk, dioleyl phosphatidyl choline, dilinoleoyl phosphatidyl choline, hydrogenated phosphatidyl choline, and phosphatidyl ethanolamine derivatives, among others; or clofibric acid derivatives, such as clofibric acid octyl amide.

In further embodiments, transdermal penetration enhancers include: 2 N-nonyl-1,3-dioxolanes; N-acetyle prolinate esters, such as, for example pentyl- and octyl-N-acetyl prolinate, among others; alkyldiloxanes, such as, for example 1-alkyl-3-b-Dglucopyranosyl-1,1,3,3-tetramethyl disiloxanes, N-arginine chitosan, dodecyl-6-(dimethylamino)hexanoate, laurocapram, decenoic acid, trypsin, transcutol, tricaprylin, oleyl pyroglutamate, 1-[2-(decylthio)ethyl]anacyclopentan-2-one, ethyl (3,6-dimethyl octyl thio)acetate, and 3,7-dimethyl octyl propionate, a combination thereof; or any other chemical known to a person skilled in the art that exhibits penetration enhancing effect on transdermal absorption.

In an example, the amount of permeation enhancers included within transdermal pharmaceutical compositions range from about 0.5% to about 50%; preferably from about 1% to about 20%. These percentages may refer to % weight by weight, % weight by volume, or % volume by volume.

Administration

In some embodiments, transdermal pharmaceutical compositions allow the delivery of testosterone and AIs directly into the patient's bloodstream bypassing the gastrointestinal tract and the hepatic metabolism. In these embodiments, transdermal pharmaceutical compositions will provide higher percentages of bioavailability of testosterone and AIs to the patient, and this also allows lower dosage requirements for testosterone.

In some embodiments, transdermal pharmaceutical compositions are applied to any area of skin, such as, for example planter foot arch, lateral ankle, palm, upper arm, ventral forearm, dorsal forearm, back, chest, thigh, abdomen, groin, scalp, axilla, forehead, lower back, buttocks or scrotum, among others. In these embodiments, most suitable sites to apply transdermal pharmaceutical compositions are ventral forearm, upper arm, and chest. In other embodiments, transdermal pharmaceutical compositions are applied to those areas of skin that provide maximal systemic absorption due to increased cutaneous blood flow and heat.

In some embodiments, transdermal pharmaceutical compositions include liquid dosage forms, such as, for example solutions, liquid sprays, lotions, and the like. In other embodiments, transdermal pharmaceutical compositions include semi-solid dosage forms, such as, for example emulsions, creams, gels, pastes, ointments, and the like.

In some embodiments, transdermal dosage forms can be designed for fast release and transdermal absorption of testosterone and AIs. In other embodiments, transdermal dosage forms can be designed for slow release and transdermal absorption of testosterone and AIs over a prolonged period of time.

In some embodiments, transdermal pharmaceutical compositions are administered in a single administration whereby a certain amount of testosterone and AIs is administered at once. In other embodiments, transdermal pharmaceutical compositions are administered by multiple administrations in one or more sub-doses over a specified period of time.

In some embodiments, transdermal pharmaceutical compositions may be tailored for individual patients according to clinical symptoms and baseline serum concentrations of testosterone and estradiol. In these embodiments, transdermal pharmaceutical compositions may be prescribed with various concentrations of testosterone and AIs and suitable dosage regimens to more closely mimic the circadian rhythm and physiological pulsatile secretion of testosterone, thereby keeping the testosterone and estradiol levels within physiologic range.

In some embodiments, the dosages (e.g., daily) required depend on the type of AI included in the disclosed transdermal pharmaceutical compositions. In other words, some AIs are more potent than others, and hence, the dosage regimen varies among the various AIs used.

In an example, transdermal pharmaceutical compositions are administered within a dosage range from about 25 mg/day to about 500 mg/day of testosterone, preferably from about 40 mg/day to about 120 mg/day; and from about 0.01 mg/day to about 1.0 mg/day of anastrozole, preferably from about 0.1 mg/day to about 0.5 mg/day.

In some embodiments, low dose APIs in any of the above identified dosage forms can result in acceptable testosterone levels in the patient. This contrasts with conventional testosterone replacement therapy that involves administering high dosages of testosterone.

The following examples are intended to illustrate the scope of the disclosure and are not intended to be limiting. It is to be understood that other pharmaceutical formulations known to those skilled in the art may alternatively be used.

### EXAMPLES

The following are exemplary of dosage forms of the transdermal pharmaceutical compositions.

Example #1 illustrates formula for a transdermal testosterone/anastrozole solution. These percentages may refer to % weight by weight, % weight by volume, or % volume by volume.

| Ingredient | Composition |
|---|---|
| Anastrozole | 0.05% |
| Testosterone | 5% |
| Penetration enhancer(s) | 1-10% |
| Other solvents | 0-30% |
| Thickening agent (optional) | 0.1-5% |
| Ethanol 190 Proof USP | q.s. 100% |

Example #2 illustrates formula for a transdermal testosterone/anastrozole gel. These percentages may refer to % weight by weight, % weight by volume, or % volume by volume.

| Ingredient | Composition |
|---|---|
| Anastrozole | 0.05% |
| Testosterone | 5% |
| Penetration enhancer(s) | 1-10% |
| Co-solvent(s) | 0-20% |
| PCCA VersaBase ® Gel | q.s. 100% |

*It is a proprietary topical gel base produced by Professional Compounding Centers of America (PCCA)

Example #3 illustrates formula for a transdermal testosterone/anastrozole cream. These percentages may refer to % weight by weight, % weight by volume, or % volume by volume.

| Ingredient | Composition |
|---|---|
| Anastrozole | 0.05% |
| Testosterone | 5% |
| Penetration enhancer(s) | 1-10% |
| Co-solvent(s) | 5-20% |
| Base, PCCA Vanishing | q.s. 100 gm |
| Cream Light ™* | |

*It is a proprietary topical cream base produced by Professional Compounding Centers of America (PCCA)

While various aspects and embodiments have been disclosed, other aspects and embodiments are contemplated. The various aspects and embodiments disclosed are for purposes of illustration and are not intended to be limiting, with the true scope and spirit being indicated by the following claims.

What is claimed is:

1. A pharmaceutical composition comprising about 2% to about 20% testosterone weight by weight and an aromatase inhibitor wherein the aromatase inhibitor is selected from the group consisting of anastrozole, letrozole, exemestane, vorozole, formestane, fadrozole, and testolactone.

2. The pharmaceutical composition of claim 1, wherein the aromatase inhibitor is anastrozole.

3. The pharmaceutical composition of claim 1, wherein the pharmaceutical composition comprises about 5% to about 10% testosterone weight by weight.

4. The pharmaceutical composition of claim 2, wherein the pharmaceutical composition comprises about 0.01% to about 0.1% anastrozole weight by weight.

5. The pharmaceutical composition of claim 4, wherein the pharmaceutical composition further comprises at least one additive selected from the group consisting of diluents, thickening agent, transdermal penetration enhancers, pH adjusters, preservatives, colors, stabilizing agents, antioxidants, and surfactants.

6. The pharmaceutical composition of claim 5, wherein the pharmaceutical composition comprises about 0.5% to about 50% of at least one transdermal penetration enhancer weight by weight.

7. The pharmaceutical composition of claim 6, wherein the pharmaceutical composition comprises about 1% to about 20% of at least one transdermal penetration enhancer weight by weight.

8. A method of treating human male testosterone deficiency comprising applying a transdermal pharmaceutical composition to skin wherein the transdermal pharmaceutical composition comprises about 2% to about 20% testosterone weight by weight and an aromatase inhibitor wherein the aromatase inhibitor is selected from the group consisting of anastrozole, letrozole, exemestane, vorozole, formestane, fadrozole, and testolactone.

9. The method of claim 8, wherein the aromatase inhibitor is anastrozole.

10. The method of claim 9, wherein the transdermal pharmaceutical composition delivers about 25 mg/day to about 500 mg/day of testosterone and about 0.01 mg/day to about 1.0 mg/day of anastrozole.

11. The method of claim 10, wherein the transdermal pharmaceutical composition comprises clomiphene citrate and wherein the transdermal pharmaceutical composition delivers about 40 mg/day to about 120 mg/day of testosterone and about 0.1 mg/day to about 0.5 mg/day of clomiphene citrate.

12. The method of claim 9, wherein the transdermal pharmaceutical composition comprises about 0.01% to about 0.1% anastrozole weight by weight.

13. The method of claim 9, wherein the transdermal pharmaceutical composition comprises about 5% to about 10% testosterone weight by weight.

14. The method of claim 12, wherein the transdermal pharmaceutical composition further comprises at least one additive selected from the group consisting of diluents, thickening agent, transdermal penetration enhancers, pH adjusters, preservatives, colors, stabilizing agents, antioxidants, and surfactants.

15. The method of claim 12, wherein the transdermal pharmaceutical composition comprises about 0.05% anastrozole.

16. The method of claim 10, wherein the transdermal pharmaceutical composition is a liquid dosage form wherein the liquid dosage form is a solution, a liquid spray, or a lotion.

17. The method of claim 10, wherein the transdermal pharmaceutical composition is a semi-solid dosage form wherein the semi-solid dosage form is selected from the group consisting of an emulsion, a cream, a gel, a paste, and an ointment.

18. The method of claim 16, wherein the transdermal pharmaceutical composition is a solution comprising about 0.01% to about 0.1% anastrozole weight by weight, about 5% to about 10% testosterone weight by weight, about 1% to about 10% of at least one penetration enhancer, and ethanol.

19. The method of claim 18, wherein the solution further comprises about 0.1 to about 5% weight by weight of thickening agent.

20. The method of claim 17, wherein the transdermal pharmaceutical composition is a gel comprising about 0.01% to about 0.1% anastrozole weight by weight, about 5% to about 10% testosterone weight by weight, and about 1% to about 10% of at least one penetration enhancer.
